(19) United States
(12) Patent Application Publication
(10) Pub. No.: US 2025/0153114 A1
(43) Pub. Date: May 15, 2025

(54) POLYIMIDE-CONTAINING FILTRATION MEMBRANE, FILTERS, AND METHODS

(71) Applicant: ENTEGRIS, INC., Billerica, MA (US)

(72) Inventors: Yasushi Oyashiki, Narashino-shi (JP); Akihiro Sakano, Yonezawa-shi (JP)

(21) Appl. No.: 19/020,885

(22) Filed: Jan. 14, 2025

Related U.S. Application Data
(63) Continuation of application No. 16/803,281, filed on Feb. 27, 2020.
(60) Provisional application No. 62/811,334, filed on Feb. 27, 2019.

(51) Int. Cl. B01D 69/10; B01D 63/14; B01D 65/00; B01D 69/02; B01D 69/06; B01D 71/64; C07C 41/36; C07C 67/56

(52) U.S. Cl. CPC B01D 69/108; B01D 63/14; B01D 69/02; B01D 69/06; B01D 71/64; C07C 41/36; C07C 67/56; B01D 65/003; B01D 2313/2061; B01D 2313/21; B01D 2325/04; B01D 2325/20; B01D 2325/24

(57) ABSTRACT

Described are filtration membranes that include a porous polyimide membrane and thermally stable ionic groups; filters and filter components that include these filtration membranes; methods of making the filtration membranes, filters, and filter components; and method of using a filtration membrane, filter component, or filter to remove unwanted material from fluid.

### CROSS REFERENCES TO RELATED APPLICATIONS

This application is a continuation of U.S. patent application Ser. No. 16/803,281, filed Feb. 27, 2020, which claims the benefit of U.S. Application No. 62/811,334, filed on Feb. 27, 2019, each of which are hereby incorporated by reference in their entirety.

### FIELD

The following description relates to porous polyimide-containing filtration membranes (“polyimide membranes” or “polyimide filtration membranes”); filters and filter components (i.e., any portion, piece, subcomponent, or structure of a filter) that include a polyimide filtration membranes; methods of making the filter components and filters; and method of using a filtration membrane, filter component, or filter that includes a polyimide membrane.

### BACKGROUND

Filtration membranes and filter products are indispensable tools of modern industry, used to separate unwanted materials from useful fluid materials. Unwanted materials include impurities and contaminants such as particles, microorganisms, and dissolved chemical species, which can be removed from a useful fluid such as: water; a liquid industrial solvent, raw material, or processing fluid; or a liquid solution that has medical or pharmaceutical value. Example filters are used for removing particles and bacteria from solutions such as buffers and therapeutic-containing solutions in the pharmaceutical industry, for processing ultrapure aqueous and organic solvent solutions for use in microelectronics and semiconductor processing, and for water purification processes. In one particular use, liquids used in photolithography steps for semiconductor processing must be processed using a filter to remove impurities.

To perform a filtration function, a filter product includes a filtration membrane that is responsible for removing the unwanted material from the fluid. The filtration membrane may, as required, be in the form of a flat sheet, which may be wound (e.g., spirally), or pleated, etc. The filtration membrane may alternatively be in the form of hollow fibers or capillaries. The filtration membrane can be contained within a housing that includes an inlet and an outlet, so that fluid that is being filtered enters through the inlet and passes through the filtration membrane before passing through the outlet.

Unwanted materials in the fluid are removed from the fluid by being captured by the filtration membrane either mechanically or electrostatically, e.g., by a sieving or a “non-sieving” mechanism, or both. A sieving mechanism is a mode of filtration by which a particle is removed from a flow of liquid by retention of the particle at a membrane pore due to mechanical interference of the pore with the particle movement. In this mechanism at least one dimension of the particle size is larger than pore size. A “non-sieving” filtration mechanism is a mode of filtration by which a filtration membrane retains a suspended particle or dissolved material contained in a liquid flowing through the filtration membrane in a manner that is not exclusively mechanical, e.g., that includes an electrostatic mechanism by which the particle or dissolved material is electrostatically attracted to and retained at the external or internal surface of the filtration membrane (depth filtration).

A filtration membrane can be a porous polymeric film that has an average pore size that can be selected based on the expected use of the filter, i.e., the type of filtration to be performed using the filter. Typical pore sizes are in the micron or sub-micron range, such as from about 0.001 micron to about 10 micron. Membranes with average pore size of from about 0.001 to about 0.05 micron are sometimes classified as ultrafiltration membranes. Membranes with pore sizes between about 0.05 and 10 microns are sometimes classified as microporous membranes.

For commercial use, a filtration membrane must also exhibit efficient and reliable filtering functionality, e.g., must be capable of efficiently removing a high amount of impurities from a continuous flow of fluid that passes through the filtration membrane. Filtering performance is normally assessed by two parameters including flux and retention. Flux assesses the rate of fluid flow through a filter or filtration membrane, and must be sufficiently high to reflect that a high level of flow through the filter is possible, hence the filter is economically viable. Retention, generally, refers to the amount (in percent) of impurities removed from a flow of fluid through a filter and is an indication of filter efficiency. Membrane flux and retention both significantly depend on the membrane microstructure. A membrane with smaller pores has a higher bubble point and a better sieving retention capability at the expense of lower flux (assuming the same membrane morphology and thickness). A larger pore size corresponds to a lower bubble point and a lower sieving retention, but a higher flux, assuming the same membrane morphology and thickness. The non-sieving retention capability of a membrane is a more complex property which depends on membrane surface properties (such as charge) in addition to membrane microstructure and pore size.

One area of major commercial interest for membrane filtration is the contamination removal from photoresist solutions in the semiconductor industry. As the semiconductor industry moves toward smaller nodes, problems with contamination become more difficult because as smaller-sized particles become potential contaminants that can create defects in a semiconductor substrate. Potential contaminants in a photoresist fluid include gels, ions, or nanoparticles of an organic or inorganic nature.

A filter useful within a larger processing system, e.g., for semiconductor device manufacturing, will include a housing that contains the filtration membrane and other non-membrane structures. The materials of the non-membrane structures should preferably be inert and have no effect on the fluid that is being processed by the filter. The non-membrane structures should not affect the fluid in any way, such as by altering the composition of the fluid by presenting a contaminant into the fluid. With continuously reduced dimensions of microelectronic device features and related processing features allowing smaller and smaller materials to become potential contaminants in semiconductor processing, materials used to prepare filter housings and other non-membrane filter structures may contribute contaminants in the form of organic materials removed from (extracted from) those materials and becoming present in a fluid passing through the filter.

Fluorinated polymers, e.g., thermoplastic fluorinated polymers, are known to be useful as a filter housing or other non-membrane filter structure. Fluoropolymers are relatively inert, and can produce levels of organic material contamination by extraction that are lower than levels produced by other polymers, such as polyolefins (e.g., polyethylene). But not all types of polymeric filtration membranes are capable of being incorporated into a filter housing made of fluoropolymer. During assembly of a filter, an end or edge of a filtration membrane must be secured to a thermoplastic fluoropolymer support surface (e.g., and “end piece”) in a manner that produces a liquid-tight seal between the end or edge and the support surface (e.g., end piece). This step, sometimes referred to as a “potting” step (also known as a “thermal bonding step”), requires the polymeric filtration membrane and the thermoplastic fluoropolymer support to be heated to a relatively high temperature to soften the fluoropolymer, e.g., at least 200, 300, or 400 degrees Celsius. Many polymers that are used for forming polymeric filtration membranes are not sufficiently heat stable to withstand a temperature reached during a thermal bonding step.

Filtration technologies, particularly in the semiconductor manufacturing industry, require continuing progress toward identifying new filtration membranes and filters that are effective in removing ever-smaller contaminants from useful fluids, and that do not result in materials (e.g., organic materials) being released from a filter structure into a fluid that is being processed for contaminant removal.

### SUMMARY

The following description relates to filter components and filters that include a polyimide-containing filtration membrane (sometimes referred to herein, for short, as a “polyimide filtration membrane” or “polyimide membrane”) secured to a thermoplastic structure (e.g., a non-membrane filter component) such as a fluoropolymer end piece. The description also relates to methods of preparing filters and filter components as described, and methods of using a filtration membrane, filter component, or filter as described.

The polyimide membranes may be useful with a filter of any type, for any purpose, but is described herein to be useful for filtering a liquid fluid used in semiconductor processing, for example a photoresist solution or a solvent of a photoresist solution. In the area of microelectronic device processing, wide varieties of liquid materials are used, many of which are used at a very high level of purity. As an example, solvents for photolithography processing of microelectronic devices must be of very high purity and, therefore, require a stable and clean filtration membrane to provide a useful source of these materials.

Liquid materials used in microelectronics processing may be highly acidic or corrosive, and commonly are used at elevated temperatures. These liquids, especially at elevated temperature, tend to dissolve or weaken many common polymeric materials used in filters, such as polyolefins and nylons. For this reason, fluorinated polymers such as poly (tetrafluoroethylene) (PTFE), which are considered to exhibit high levels of chemically inertness and thermal stability, are often used in filters for processing liquid materials used in microelectronic device processing.

Various commercial uses can benefit from performance properties of filters as described herein, including: photoresist chemical dispense system for semiconductor, LCD flat panel display, hard disc drive, OLED (organic light emitting diode) semiconductor structures, and other electronics device manufacturing industries; organic solvent filtration for semiconductor, LCD flat panel display, hard disc drive, OLED and other electronics device manufacturing industries; photoresist chemical manufacturing processes used by chemical company suppliers of these specific chemical formulations; organic solvent purification and supply systems; and high purity organic solvent manufacturing processes.

According to the present description, a filter or filter component is made using a polyimide-containing filtration membrane, and fluoropolymer materials for non-membrane structures. The polyimide membrane is temperature stable at temperatures required for a potting step to secure the membrane to a thermoplastic fluoropolymer support piece such as an end piece. The polyimide membrane also exhibits useful or advantageous filtering properties such as a useful level of flow (e.g., flux) of liquid that may be flowed through the membrane, and good or advantageous particle removal efficiency (e.g., “retention”). Fluoropolymer materials used for the non-membrane filter structures produce a lower level of organic materials extracted into a liquid passing through the filter, compared to other types of polymeric filter housings.

As presented herein, membranes made of a polyimide-containing polymer can be useful as a filtration membrane because these types of polymer may exhibit excellent chemical compatibility, including with many organic solvents commonly used for photolithography applications on semiconductor manufacturing process. Polyimide membranes can also be prepared with structures that produce excellent particle removal performance by sieving and non-sieving (adsorption) mechanisms, and these polymers can exhibit high tensile strength.

In one aspect, a filter component includes: a porous filtration membrane comprising polyimide polymer and having an edge; and a support piece comprising thermoplastic fluoropolymer. The edge is thermally bonded to the support piece to provide a fluid-tight seal between the edge and the support piece.

In another aspect, a method of preparing a filter component includes a porous filtration membrane in contact with thermoplastic fluoropolymer. The porous filtration membrane includes polyimide polymer and has an edge. The method includes heating the thermoplastic fluoropolymer to soften the thermoplastic fluoropolymer.

### BRIEF DESCRIPTION OF THE DRAWINGS

FIG. 1A shows an exemplary multi-layer structure containing a polyimide membrane as described.

FIGS. 1B and 1C show end views of exemplary filter components as described.

FIG. 1D is a side perspective view of an exemplary filter component as described, including a filtration membrane thermally bonded to an end piece.

FIG. 2 is a cut-away view of an example filter as described.

FIG. 3 is a table with data relating to linear hydrocarbon leaching.

FIGS. 4A and 4B are tables with data relating to organic extractables and metal extractables.

FIG. 5 is a table with data relating to particle retention.

The drawings are schematic, are not to scale, and are not be considered to limit any aspect of the present description.

### DETAILED DESCRIPTION

Described herein are filter components and filters that include a polyimide-containing filtration membrane (sometimes referred to herein, for short, as a “polyimide filtration membrane” or “polyimide membrane”). Methods of preparing filters and filter component as described, and methods of using a filtration membrane, filter component, or filter as described are also disclosed.

The filter component includes a polyimide filtration membrane and a fluoropolymer (e.g., thermoplastic fluoropolymer) support structure such as an end piece, to which the polyimide filtration membrane is attached. The polyimide membrane is secured to the fluoropolymer support structure by a potting step that heats both of the polyimide membrane and the fluoropolymer support structure to a potting temperature (of at least 200 degrees C.), to produce a fluid-tight seal between an edge of the polyimide filtration membrane and the fluoropolymer support structure (e.g., end piece).

The polyimide filtration membrane can be a porous membrane that may be in the form of a flat planar sheet, a flat disk, a pleated sheet, a wound sheet, a hollow fiber membrane, or another form of a porous filtration membrane that may be incorporated into a filter component or filter as described. The polyimide filtration membrane can exhibit physical and chemical properties that allow the polyimide membrane to be effective as a filtration membrane for processing (filtering) organic solvents to a very high level of purity, on a commercial scale.

Polyimides (sometimes abbreviated PI) are polymers that include imide linkages. A polyimide polymer may optionally contain chemical linkages in addition to imide linkages, such as amide linkages. Polymers that contain imide and amide linkages are referred to as “polyimide-polyamide” polymers. Polymers that do not contain amide linkages or other non-imide linkages (e.g., ester linkages, ether linkages) are referred to as “pure” polyimides; these polymers contain imide linkages but no ester, amide, or ether linkages, or may contain an insubstantial amount thereof relative to imide linkages, such as less than 5, 2, or 1 percent total ester, ether, and amide linkages per total amount of imide linkages. When the term “polyimide” is used herein, it refers collectively to both pure polyimides and polyimide-polyamide polymers.

Polyimides and polyimide-amides can be prepared by known methods, including by reacting combinations of monomers that include a diamine and a dianhydride, to produce a polymer having multiple polyimide linkages. By alternative routes, these materials may be made by reacting diisocyanate monomers with dianhydride monomers.

As appreciated now by Applicant, a polyimide that contains the imide linkages along with aromatic groups distributed along the polymer can exhibit useful non-sieving filtering properties, for example with polar particles or gels, which are types of particles sometimes found in solvents for processing microelectronic devices, semiconductor devices, for example in photoresist solutions used in photolithography processes. Thus, example polyimide polymers be made to include aromatic groups in combination with imide linkages; i.e., example polyimide polymers include aromatic polyimides.

Useful or preferred polyimides can be prepared from monomers that include aromatic functionality, so that the polyimide will include aromatic functionalities along the polymer chain. Monomers effective to provide a polyimide that includes aromatic functionality include aromatic diamines and aromatic dianhydrides. Aliphatic diamines can also be useful either alone or in combination with aromatic diamine.

Example aromatic diamines include aromatic diamino compounds such as diamino phenyl compounds, diamino diphenyl compounds, and the like. More specific examples include phenylenediamine and derivatives thereof, diaminobiphenyl compounds and derivatives thereof, diaminodiphenyl compound and derivatives thereof, diamino triphenyl compound and derivatives thereof, diaminonaphthalene and derivatives thereof, amino-phenyl-aminoindan and derivatives thereof, diamino tetraphenyl compound and derivative thereof, diamino hexa phenyl compounds and derivatives thereof, and cardo fluorene diamine derivative.

Example phenylenediamine compounds include m-phenylenediamine, p-phenylenediamine, and phenylenediamine derivatives having an attached alkyl group such as an ethyl or a methyl group, e.g., 2,4-diaminotoluene and the like.

Examples of diaminobiphenyl compounds include 4,4′-diaminodiphenyl, 4,4′-diamino-2,2′-bis (trifluoromethyl) biphenyl, and the like.

Diaminodiphenyl compounds are those having two aminophenyl groups bonded with each other via another (connecting) group such as an ether, a sulfonyl, a thioether, an alkylene group, an imino group, an azo group, a phosphine oxide group, an amide bond, a ureylene bond, and the like.

Example diaminodiphenyl compounds include: 3,3′-diaminodiphenyl ether, 3,4′-diaminodiphenyl ether, 4,4′-diaminodiphenyl ether, 3,3′-diaminodiphenyl sulfone, 3,4′-diaminodiphenyl sulfone, 4, 4′-diaminodiphenyl sulfone, 3,3′-diaminodiphenylmethane, 3,4′-diaminodiphenylmethane, 4,4′-diaminodiphenylmethane, 4,4′-diaminodiphenyl sulfide, 3,3′-diaminodiphenyl ketone, 3, 4′-diaminodiphenyl ketone, 2,2-bis (p-aminophenyl) propane, 2,2′-bis (p-aminophenyl) to hexa fluoro propane, 4-methyl-2,4-bis (p-aminophenyl)-1-pentene, 4-methyl-2,4-bis (p-amino phenyl)-2-pentene, 4-methyl-2,4-bis (p-aminophenyl) pentane, bis (p-aminophenyl) phosphine oxide, 4,4′-aminoazobenzene, 4,4′-diaminodiphenyl urea, 4,4′-diaminodiphenyl amide, 1,4-bis (4-aminophenoxy) benzene, 1,3-bis (4-aminophenoxy) benzene, 1,3-bis (3-amino phenoxy) benzene, 4,4-bis (4-aminophenoxy) biphenyl, bis [4-(4-aminophenoxy) phenyl] sulfone, bis [4-(3-aminophenoxy) phenyl] sulfone, 2,2 bis [4-(4-aminophenoxy) phenyl] propane, 2,2-bis [4-(4-aminophenoxy) phenyl], and the like.

Diamino triphenyl compound include two aminophenyl groups and one phenylene group, each linked via another group such as an ether, a sulfonyl, a thioether, an alkylene group, an imino group, an azo group, a phosphine oxide group, an amide bond, a ureylene bond, and the like. Examples include 1,3-bis (m-aminophenoxy) benzene, 1,3-bis (p-aminophenoxy) benzene, 1,4-bis (p-aminophenoxy) benzene, and the like.

Examples of diaminonaphthalene compounds include 1,5-diaminonaphthalene, 2,6-diaminonaphthalene and the like.

Examples of aminophenyl-aminoindan compounds include 5 or 6-amino-1-(p-aminophenyl)-1,3,3-trimethyl indane, and the like.

Examples of diamino tetraphenyl compounds include 4,4′-bis (p-aminophenoxy) biphenyl, 2,2′-bis [p-(p′-aminophenoxy) phenyl] propane, 2,2′-bis [p-(p′-aminophenoxy) biphenyl] propane, 2,2′-bis [p-(m-aminophenoxy) phenyl] benzophenone, etc.

Example cardo fluorene diamine derivatives include 9,9-bis aniline fluorene, and the like.

Example aliphatic diamines include those containing about 2 to 15 carbon atoms, such as pentamethylenediamine, hexamethylene diamine, and the like.

Useful dianhydride monomers can be aromatic or aliphatic. Examples generally include aromatic tetracarboxylic acid dianhydride compounds and aliphatic tetracarboxylic dianhydride compounds.

Examples of aromatic tetracarboxylic dianhydrides include pyromellitic dianhydride, 1,1-bis (2,3-carboxyphenyl) ethane dianhydride, bis (2,3-carboxyphenyl) methane dianhydride, bis (3,4-carboxyphenyl) methane dianhydride, 3,3′, 4,4′-biphenyltetracarboxylic dianhydride, 2,3,3′, 4′-biphenyltetracarboxylic dianhydride, 2,2,6,6-biphenyltetracarboxylic dianhydride, 2,2-bis (3,4-carboxyphenyl) propane dianhydride, 2,2-bis (2,3 dicarboxyphenyl) propane dianhydride, 2,2-bis (3,4-carboxyphenyl)-1,1,1,3,3,3-hexa fluoro propane dianhydride, 2,2-bis (2,3-carboxyphenyl)-1,1,1,3,3,3-hexafluoropropane dianhydride, 3,3′, 4,4′-benzophenonetetracarboxylic dianhydride, bis (3,4-carboxyphenyl) ether dianhydride, bis (2,3-carboxyphenyl) ether dianhydride, 2,2′, 3,3′-benzophenone tetracarboxylic dianhydride, 4,4-(p-phenylene-oxy) diphthalic anhydride, 4,4-(m-phenylenedioxy) diphthalic dianhydride, 1,2,5,6-naphthalene tetracarboxylic dianhydride, 1,4,5,8-naphthalene tetracarboxylic dianhydride, 2,3,6,7-naphthalene tetracarboxylic dianhydride, 1,2,3,4-benzene tetracarboxylic dianhydride, 3,4,9,10-perylenetetracarboxylic dianhydride, 1,2,7,8-phenanthrene tetracarboxylic acid dianhydride, 9,9-bis phthalic anhydride fluorene, 3,3′, 4,4′-diphenylsulfone tetracarboxylic dianhydride anhydride, and the like.

Examples of the aliphatic tetracarboxylic dianhydrides include ethylene tetracarboxylic dianhydride, butane tetracarboxylic dianhydride, cyclopentane tetracarboxylic dianhydride, cyclohexane tetracarboxylic dianhydride cyclohexane, 1, 2, 4, 5 hexanoic acid dianhydride cyclohexane, 1,2,3,4-cyclohexane tetracarboxylic acid dianhydride, and the like.

Another desirable property of a polyimide for use in a filtration membrane is high mechanical strength, e.g., tensile strength. A useful or preferred polyimide useful for a filtration membrane as described can exhibit a tensile strength (machine direction) of at least 1000, 2500, or 4000 mN per 5 mm and a tensile strength (cross direction) of at least 1000, 2500, or 4000 mN per 5 mm (e.g., as measured suing a Shimadzu AGS-H autograph at 20 mm/min of cross-head speed, Load cell 100N.

Examples of commercial polyimides include polymers sold by DuPont under that trade name Kapton®, and polymers sold by Dupont, and polyimides sold by Tokyo Ohka Kogyo Co. Ltd.

A “polyimide membrane,” as that term is used herein, refers to a porous (e.g., microporous, ultraporous, etc.) filtration membrane having physical properties and filtering performance properties as described, and that includes a useful or a high amount of polyimide as described (including pure polyimide polymers and polyimide-polyamide polymers). If desired, but not necessarily preferred, the polyimide membrane may be made of a blend of polyimide and one or more other polymers. A useful polyimide membrane may comprise, consist of, or consist essentially of polyimide. For example, a polyimide membrane may include polyimide polymer blended with another polymer such as a thermoplastic polyolefin (e.g., polyethylene or polypropylene), a nylon, polysulfones, or a fluoropolymer. In specific examples, the polyimide membrane can be made of mostly polyimide polymer, e.g., at least 70, 80, 90, 90, 98, or 99 weight percent polyimide polymer. A porous polyimide membrane that consists essentially of polyimide (including polyimide-polyamide) is a membrane that contains only polyimide and not more than 2, 1, 0.5, or 0.1 weight percent of any other type of polymer.

In some embodiments, polyimide materials have been identified for use in a filter as described, as a filtration membrane, in combination with a fluoropolymer support piece (e.g., end piece), due in part to properties of polyimides that can provide for useful or advantageous processing and performance properties of the combination. Specific properties of polyimide polymers that can be particularly well-suited for use as a filtration membrane include: desired tensile strength; high thermal stability and high chemical stability, i.e., good resistance to high temperatures and good resistance to chemical degradation; and the ability to form polyimide into a porous filtration membrane that exhibits useful or advantageous filtering performance (e.g., flow time, bubble point, retention).

A particular advantage of polyimide as a membrane material is a high thermal stability, which allows a polyimide membrane to be processed to form a filter or filter component that includes other pieces made of fluoropolymer. The polyimide and the polyimide membrane can be thermally stable for processing the polyimide membrane to form a filter component, such as for attaching an edge of the polyimide membrane to another piece of a filter component that is made of thermoplastic fluoropolymer, such as an end piece or other support. Sufficient thermal stability refers to a polyimide membrane that is stable at a temperature useful in a potting step to secure the membrane to a thermoplastic end piece, e.g., the membrane will retain desired physical and filtering performance properties if heated to a temperature of a potting step. Example such temperatures can be at least 200 degrees Celsius, e.g., at least 250 or 300 degrees Celsius, or even at least 400 or 500 degrees Celsius.

In more detail, a polyimide filtration membrane as described is sufficiently stable upon heating to withstand processing steps used to convert the polyimide filtration membrane into a filter component or a finished filter by steps that include securing an edge of the membrane to a thermoplastic fluorinated end piece by heating the end piece to soften or melt the thermoplastic fluorinated end piece, e.g., a “potting” step. A potting step commonly used to secure a filtration membrane into a non-membrane filter structure, e.g., to secure an edge of the filtration membrane to a surface of an end piece (or other support structure) of a filter by use of a thermally-processable fluoropolymer, which may be the material of the end piece. To perform the potting step, the thermoplastic fluoropolymer (e.g., end piece) may be heated to a temperature at which the thermoplastic fluoropolymer becomes sufficiently soft or melted to allow the fluoropolymer to contact an edge of a membrane (with pressure) and become securely bonded to the edge of the membrane and to form a fluid-tight seal between the edge and the end piece. The required temperature depends on the type of thermoplastic fluoropolymer that is used, and may be at least 200 degrees Celsius, e.g., at least 250 or 300 degrees Celsius, or even at least 400 or 500 degrees Celsius.

A polyimide filtration membrane as described is porous, having an “open pore” structure that allows for a desired flow of fluid (e.g., liquid) from one side or surface of the filtration membrane, through a thickness of the filtration membrane, to exit the opposite side or surface of the filtration membrane. Between the two opposed surfaces, along the thickness of the membrane, are cellular, three-dimensional, void microstructures in the form of enclosed cells, i.e., “open cells” or “pores” that allow liquid fluid to pass through the thickness of the membrane. The open cells can be referred to as openings, pores, channels, or passageways, which are largely interconnected between adjacent cells to allow the liquid fluid to flow through the cells, between the cells, and through the thickness of the polyimide filtration membrane from a first side to exit at a second, opposite side.

Physical properties of a filtration membrane that are relevant to filtering performance include porosity, thickness, and pore size, which relate to desired properties of bubble point, filtering efficiency (e.g., measured by “retention”), and flow rate (or flux) through the filtration membrane (e.g., as measured by flow time).

Examples of useful polyimide membranes as described can be in the form of a sheet, which may be optionally flat, folded (e.g., pleated) or wound when incorporated into a filter component or filter. The sheet may be of any useful thickness, with useful or preferred examples being in a range from 5 to 100 microns, e.g., from 10 to 80 microns, or from 20 to 50 microns.

The membrane can have a porosity that allows the membrane to be effective as described herein, to allow a suitable flow rate of liquid to pass through the membrane while also removing a high level of contaminants or impurities from the liquid. Examples of useful membranes can have a porosity of up to 80 percent, e.g., a porosity in a range from 60 to 80, e.g., 60 to 70 percent or from 40 to 60 percent. As used herein, and in the art of porous bodies, a “porosity” of a porous body (also sometimes referred to as “void fraction”) is a measure of the void (i.e. “empty”) space in the body as a percent of the total volume of the body, and is calculated as a fraction of the volume of voids of the body over the total volume of the body. A body that has zero percent porosity is completely solid.

A pore size that will be useful for a particular polyimide membrane can depend on factors such as: the thickness of the membrane; the desired flow properties (e.g., flow rate or “flow time”) of fluid through the membrane; desired level of filtering (e.g., as measured by “retention”); the particular type of fluid that will be processed (filtered) by passing through the membrane; the particular contaminant that will be removed from the fluid passing through the membrane; as well as other factors. For certain presently understood examples, useful pore sizes may be in a range from about 10, 20, 30, or 40 nanometers, up to about 4, 8, or 10 microns, including ranges of pore sizes sometimes classified as “microporous,” “ultraporous,” or “nanoporous.” The term “microporous” is sometimes used to refer to pores within any of these size ranges, including microporous and sub-microporous sizes, as a way of distinguishing from materials having larger pore sizes, i.e., to distinguish from materials that are considered to be “macroporous.” Pore size is often reported as average pore size of a porous material, which can be measured by known techniques such as by Mercury Porosimetry (MP), Scanning Electron Microscopy (SEM), Liquid Displacement (LLDP), or Atomic Force Microscopy (AFM).

Pore size of a membrane may also be assessed based on a correlation to the property known as “bubble point,” which is an understood property of a porous filtration membrane. Bubble point corresponds to pore size, which may also correspond to filtering performance, e.g., as measured by retention. A smaller pore size can correlate to a higher bubble point and often to higher filtering performance (higher retention). Normally, however, a higher bubble point also correlates to relatively higher resistance of flow through a porous material, and a higher flow time (lower rate of flow for a given pressure drop). Example filtration membranes of the present description can exhibit a combination of a relatively higher bubble point, useful or advantageous filtering performance, and a useful level of flow, e.g., a flow rate that allows for the filtration membrane to be used in a commercial filtering process.

By one method of determining a bubble point of a porous material, a sample of the porous material is immersed in and wetted with a liquid having a known surface tension, and a gas pressure is applied to one side of the sample. The gas pressure is gradually increased. The minimum pressure at which the gas flows through the sample is called a bubble point. Examples of useful bubble points of a porous polyimide membrane useful according to the present description, measured using ethoxy-nonafluorobutane (HFE 7200), at a temperature of 20-25 degrees Celsius (e.g., 22 degrees Celsius), can be in a range from 10 to 300 pounds per square inch (psi), e.g., in a range from 20 to 200 or from 30 to 150 psi.

Advantageously, a porous membrane made using polyimide can be prepared to achieve a bubble point that is greater than a bubble point of a comparable (non-polyimide) membrane. As a specific example, a thermally stable polyimide as described can be prepared to achieve a bubble point that is higher than a bubble point of a comparable filtration membrane made of fluoropolymer, e.g., poly (tetrafluoroethylene) (PTFE) or another fluoropolymer or perfluoropolymer commonly used as a material of a porous filtration membrane. A relatively higher bubble point of a porous membrane may be desired or advantageous for filtering performance due to a generally greater degree of particle or contaminant removal by a membrane having a higher bubble point; the membrane should still have desirable flow properties, e.g., as measured by flow rate or flow time.

Another measure of filtering performance, this one involving the level of effectiveness of a filtration membrane in removing unwanted material (i.e., “contaminants”) from a liquid, is referred to as “retention.” Retention, with reference to the effectiveness of a filtration membrane (e.g., a filtration membrane as described), generally refers to a total amount of an impurity (actual or during a performance test) that is removed from a liquid that contains the impurity, relative to the total amount of the impurity that was in the liquid upon passing the liquid through the filtration membrane. The “retention” value of a filtration membrane is, thus, a percentage, with a filter that has a higher retention value (a higher percentage) being relatively more effective in removing particles from a liquid, and a filter that has a lower retention value (a lower percentage) being relatively less effective in removing particles from a liquid.

In example embodiments of polyimide membranes as described, a membrane can exhibit a retention that exceeds 80 or 90 percent for a monolayer coverage of 1.0 percent, as measured using the test described in the Examples section, with a useful flow rate through the membrane, preferably a retention that exceeds 95, 98, or 99 percent for a monolayer coverage of 1.0 percent. Additionally or alternately, a membrane can exhibit a retention that exceeds 80 or 90 percent for a monolayer coverage of 2.0 percent, as measured using the test described in the Examples section, with a useful flow rate through the membrane, preferably a retention that exceeds 92 or 95 percent for a monolayer coverage of 2.0 percent.

Also, for comparison to previous filters and filtration membranes, a polyimide membrane can have a greater removal efficiency (as measured by retention) in comparison to a comparable fluoropolymer filter, given similar physical features of the two membranes such as membrane thickness, porosity, morphology, etc., but with the polyimide membrane having a smaller pore size and higher bubble point; for example, a polyimide membrane can have a removal efficiency (as measured by retention) that is at least 10 or 20 percent greater than a removal efficiency of a comparable fluoropolymer filter at a monolayer coverage of 1.0 percent, alternately or additionally a removal efficiency (as measured by retention) that is at least 15, 20, 25, or 30 percent greater than a removal efficiency of a comparable fluoropolymer filter at a monolayer coverage of 2.0 percent.

In combination with a desired bubble point and filtering performance (e.g., measured by retention) a membrane as described can exhibit a useful (commercially acceptable) level of a resistance to flow of liquid through the membrane. A resistance to liquid flow can be measured in terms of flow rate or flow time (which is an inverse to flow rate). A polyimide membrane as described can preferably have a useful or a relatively low flow time, preferably in combination with a bubble point that is relatively high, as well as exhibiting good or advantageous filtering performance (e.g., as measured by retention). An example of a useful or preferred flow time (i.e., “IPA flow time”) can be below about 60,000 seconds/500 milliliter, e.g., below about 50,000 or 40,000 or 20,000 seconds per 500 ml; “IPA flow time” is measured as the time it takes for 500 ml of isopropyl alcohol (IPA) fluid to pass through a membrane with a surface area of 13.8 cm2 at 14.2 psi, and at a temperature 21 degrees Celsius.

The polyimide membrane can be used in a filter (e.g., as a component of a filter cartridge) that contains the filtration membrane through which fluid can be passed to allow or cause undesired material within the fluid to be removed from the fluid by the membrane. A “filter” refers to a structure that contains the filtration membrane and additional (optional) structures such as a frame, housing, optional cylindrical core, support, lamination film, flow control structures, or the like that together allow fluid to be directed through the filter, while passing through the filtration membrane, for the filtration membrane to function to filter unwanted material from the fluid. These structures of the filter are sometimes referred to herein as “non-membrane filter structures.”

An example filter can include a housing with an inlet and an outlet, and with a polyimide filtration membrane as described contained within the housing and located between the inlet and the outlet. The polyimide membrane can be located and sealed within the housing in a manner to require that some or all of a fluid that enters the filter inlet flows through the filtration membrane before passing through the outlet of the housing to leave the filter. Within the housing, the filtration membrane may take any shape or form, such as a hollow filtration membrane, disk-shaped membranes, or a sheet-like membrane that may be wound or pleated.

The filtration membrane may be contained within the filter structure by various additional materials and structures that support or contain (house) the filtration membrane within the filter and cause fluid to flow through the filtration membrane when passing through the filter, i.e., non-membrane filter structures. Examples of such non-membrane filter structures for a filter that includes a cylindrical pleated filtration membrane include the following, any of which may be included in a filter construction but may not be required: a rigid or semi-rigid core that supports a cylindrical pleated filtration membrane at an interior opening of the cylindrical pleated filtration membrane; a rigid or semi-rigid cage that supports the cylindrical pleated filtration membrane at an exterior of the pleated membrane; a seaming material that connects the longitudinal edges of the pleated filtration membrane along a length-wise seam of the cylindrical membrane to form the membrane into a pleated cylinder; one or more fenestrated membrane support materials (e.g., in the form of a fenestrated net or mesh) that support one or both major surfaces of the filtration membrane through which fluid flows, but that is not required to be effective as a filtering material; end pieces (or “end plates or “pucks”) that are situated at each of the two opposed pleated ends of the pleated cylindrical filtration membrane; an optional (not required) potting compound in the form of a melt-processable fluoropolymer that can be used for thermally bonding a pleated edge of the filtration membrane to an end piece; and a lamination film, which is located at the opposed pleated end edges of the cylindrical pleated membrane where the edges meet the end pieces.

According to useful and preferred embodiments of a filter cartridge and a filter as described, the components of the filter other than the polyimide membrane may be made of fluoropolymer, e.g., perfluorinated polymer, including but not necessarily thermoplastic fluoropolymer. Each non-membrane filter structure may be either fluorinated (at least partially fluorinated) or perfluorinated (substantially fully fluorinated).

A perfluorinated polymer (“perfluoropolymer”), based on common terminology, is a polymer in which all or substantially all (e.g., at least 95, 98, or 99 percent) of hydrogen atoms of the polymer are replaced by fluorine atoms. A fluorinated polymer (“fluoropolymer”), based on common terminology, is a polymer that has a carbon backbone that has fluorine atoms as replacements for hydrogen atoms, but that can also include a more than insubstantial amount of hydrogen atoms, chlorine atoms, or both, attached directly to the carbon backbone, with the fluorine atom content being sufficiently high (e.g., 50, 60, 70, or 80 percent) to provide the polymer with desired heat and chemical stability properties.

Examples of fluorinated and perfluorinated polymers useful as a component of a filter cartridge or filter as described include poly (tetrafluoroethylene) (PTFE), poly (tetrafluoroethylene-co-hexafluoropropylene) (FEP), poly (tetrafluoroethylene-co-perfluoro (alkylvinyl ether)) (PFA), poly (ethylene-co-tetrafluoroethylene) (ETFE), poly (chlorotrifluoroethylene) (CTFE), poly (chlorotrifluoroethylene-co-ethylene) (ECTFE), polyvinylidene fluoride (PVDF), and polyvinyl fluoride (PVF).

According to preferred filters of the present description, a filter can be made partly, mostly, entirely, or substantially entirely of the polyimide membrane supported by non-membrane filter structures that are made of fluorinated (e.g., perfluorinated) polymeric materials, each of which, independently, may also be “thermally-processable,” i.e., “thermoplastic.” Preferred filters can be made entirely of non-membrane filter structures that are fluorinated materials or perfluorinated materials, meaning that the at least 90, 95, 98, 99, or 100 percent of non-membrane filter structures are fluorinated or perfluorinated.

Also, for comparison to alternative filters products that are made to include other (non-fluorinated) types of polymers as non-membrane filter structures, fluorinated materials for these structures can advantageously produce a lower amount of organic materials (e.g., linear hydrocarbon materials) being extracted from the non-membrane filter structure (i.e., hydrocarbon leaching or linear hydrocarbon leaching) into a liquid fluid passing through the filter. As specific comparisons, a filter that contains non-membrane filter structures made entirely of fluoropolymers can exhibit a substantially lower amount of (e.g., 20, 40, 50, 70, or even an 80 percent reduction of) linear hydrocarbons being extracted during use or testing as compared to comparable filter products that are made of non-membrane filter structures prepared from other (non-fluorinated) polymers such as polyolefins, including polyethylene or polypropylene.

Considered in a different manner, certain preferred filter products can be constructed so that all surfaces of a filter that will contact a liquid fluid as the liquid fluid passes through the filter is made of a fluorinated of perfluorinated material. These non-membrane filter structures include required and optional components such as a core, cage, seaming material, a polymeric (e.g., thermoplastic) “lamination film” at an edge of a membrane, membrane support material such as a net extending across one or both surfaces of the membrane, polymeric end pieces, as well as any other components of the filter structure such as flow control surfaces, gaskets, adhesives, sealants, grommets, inlets, outlets, housing components, etc. Filters that are made entirely of fluorinated non-membrane filter structures, or that contain fluorinated structures and surfaces at all locations that contact fluid that passes through the filter, are sometimes referred to “all Teflon,” or “all fluoropolymer” filters. These filters can be considered to have non-membrane filter structures that consist of or consist essentially of fluoropolymer materials, e.g., that consist of or consist essentially of perfluoropolymer materials. A filter (or filter component) that contains non-membrane filter structures that consists essentially of fluoropolymer materials or perfluoropolymer materials is a filter (or filter component) that contains non-membrane structures that are made of at least 98, 99, or 99.5 percent by weight fluoropolymer or perfluoropolymer materials (or a combination thereof), and not more than 2, 1, or 0.5 weight percent of non-fluorinated materials or structures, based on total weight of the non-membrane filter structures.

Certain non-membrane filter structures are preferably thermally-processable (i.e., “melt-processable” or “thermoplastic”), including end pieces of a filter cartridge or a filter to which an edge of a polyimide membrane is secured by a potting step. A thermally-processable fluoropolymer is a fluorinated (e.g., either partially fluorinated or fully fluorinated (perfluorinated)) polymer that is capable of reversibly softening or melting to become pliable or flowable when heated to a temperature above a softening temperature characteristic of the polymer material, and that will re-solidify when cooled to a temperature below the softening temperature. Preferred thermally-processable fluoropolymers may be heated to reversibly soften or melt, then cooled and re-solidified, repeatedly, without substantial degradation of the fluoropolymer. Specific examples of melt-processable fluoropolymers include poly (tetrafluoroethylene-co-hexafluoropropylene) (FEP) and poly (tetrafluoroethylene-co-perfluoro (alkylvinyl ether)) (PFA).

According to useful and preferred methods of preparing a filter or filter cartridge by securing an edge of a polyimide membrane to an end piece (or other support structure or a potting compound (e.g., adhesive)), the end piece (or other structure) can be a thermoplastic fluoropolymer that softens or melts at a temperature of 200 degrees Celsius or greater. The thermoplastic fluoropolymer may preferably be the material of the end piece to which the edge of the polyimide membrane is attached during the potting step. Alternately or in addition, but not necessarily, an additional thermoplastic material such as a thermoplastic potting compound (which may be a fluoropolymer as described herein) may also be placed between the edge and the end piece. According to certain presently-useful embodiments, however, a thermoplastic potting compound is not required and may specifically be excluded.

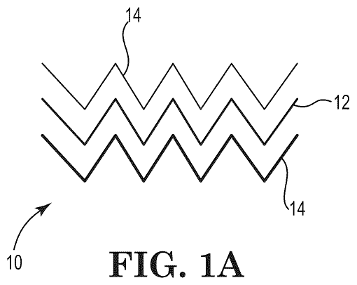

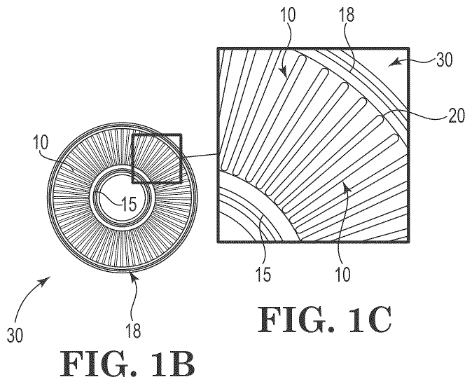

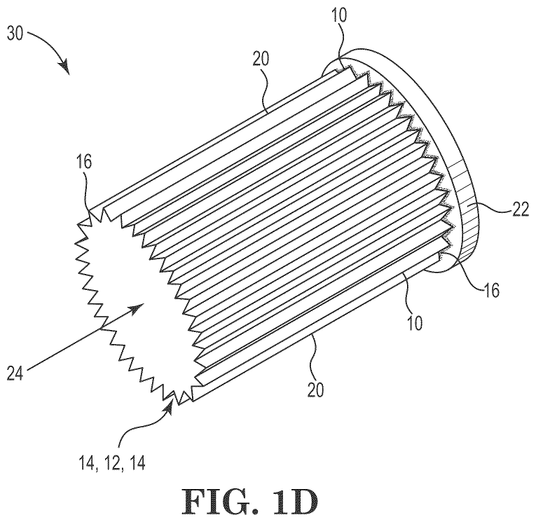

Referring now to FIG. 1A, illustrated is a single, non-limiting example of a filtration membrane as described herein, in the form of a pleated sheet-style membrane for use as part of a filter component. Filter component 10 includes polyimide filtration membrane 12 as described herein. Placed against each of the two opposed major surfaces of filtration membrane 12 is membrane support material 14 (a support piece), which is preferably a fluoropolymer mesh or netting material (e.g., a perfluoropolymer material such as PFA). Along opposed edges of membrane 12 and support material 14, at each of two opposed pleated ends of the combined layers, may optionally be amounts of lamination film (not shown) placed along the ends to hold the edges of the separate layers together. The lamination film can be made of a fluoropolymer, preferably a melt-processable fluoropolymer material (e.g., perfluoro-ethylene-propylene polymer (FEP), PFA, or the like).

Referring to FIGS. 1B and 1C, these show a cross sectional end-view (with FIG. 1C in close-up) of a filter component 30 that includes pleated, multi-layer cylindrical component 10 made with filtration membrane 12 and support materials 14 processed to form a pleated cylinder comprising pleats 20, in a longitudinal direction. After pleats 20 are formed, opposed longitudinal (non-pleated) edges of the multi-layer film structure are brought together to form the pleated cylinder and adhered together by use of a seaming material (not shown), which may be a melt-processable fluoropolymer material such as a fluorinated adhesive or polymer, e.g., a melt-processable perfluoropolymer material such as PFA.

FIG. 1C shows filter component 30, which is a product of pleated, multi-layer cylindrical component 10, cage 18, core 15, and two opposed thermoplastic fluorinated end pieces (not shown) (see FIG. 1D). Cage 18 can preferably be a fluoropolymer material, such as PFA. Core 15 can also preferably be a fluoropolymer material, such as PFA.

FIG. 1D shows a side perspective view of a filter component 30 without cage 18 or core 15, including only pleated, multi-layer cylindrical component 10 made with filtration membrane 12 and support materials 14, with one pleated edge attached to end piece 22 (a support piece) by a potting step. End piece 22 can preferably be made of a melt-processable fluoropolymer material (e.g., melt-processable perfluoropolymer material such as PFA). The step of potting the pleated edge of pleated, multi-layer cylindrical component 10 to end piece 22 includes heating cylindrical component 10 and end piece 22 to a temperature that will soften the melt-processable fluoropolymer material of end piece 22, and optional lamination film 16 (also made of melt-processable fluoropolymer material) at opposed pleated ends, and pressing the pleated end into a softened or melted surface of end piece 22. The heating temperature, contact pressure, and amount of time for the potting step can be sufficient to allow softening or melting of the melt-processable fluoropolymer materials, and flow of the fluoropolymer materials relative to the edge at the pleated end of cylindrical component 10 that are sufficient to cause the entire edge of filtration membrane 12 to become covered or permeated with melt-processable fluoropolymer, to produce a “liquid-tight” seal along the edge that will not allow fluid (e.g., liquid) to pass around the edge between the edge and the adjacent surface of end piece 22, i.e., to produce a fluid-tight (especially a liquid tight) seal at the location of the pleated end that has been thermally bonded to the surface of end piece 22.

Still referring to FIG. 1D, other steps of converting a filtration membrane 12 into a filter component or a filter including placing a cylindrical core (e.g., 15, not shown) at interior opening 24 of pleated cylindrical component 10, and a cylindrical cage (e.g., 18, not shown) about the exterior of pleated cylindrical component 10, e.g., before a potting step.

Yet another additional step can be to thermally bond a second end piece (not shown) to the second pleated end of pleated cylindrical component 30 of FIG. 1D. The second end piece can also be a fluorinated thermoplastic polymer. The resultant pleated cylindrical component, with two pleated ends secured by potting to thermoplastic fluoropolymer end pieces, to form opposed fluid-tight seals, and the optional core and cage pieces, can then be placed into a filter housing that includes an inlet and an outlet and that is configured so that an entire amount of a fluid entering the inlet must necessarily pass through filtration membrane 12 before exiting the filter at the outlet.

According to one useful series of steps, a filtration membrane as described, and optional fluoropolymer support layer, can first be processed to heat laminate two opposed edges of a sheet of the material, using a thermoplastic fluoropolymer FEP as a laminating film. That filtration membrane and optional support layer (or layers), with heat laminated edges, are then pleated and the pleated membrane is seamed along the non-pleated edges into a cylindrical “pleat pack” using a thermoplastic fluoropolymer such as FEP to connect the remaining two (non-pleated) edges. A thermoplastic fluoropolymer (e.g., PFA) cylindrical core structure is inserted into the middle of the pleat pack, and the pleat pack is inserted into a cylindrical fluoropolymer (e.g., PFA) cage. This assembly (or “cartridge”) is ready to be thermally bonded to two thermoplastic fluoropolymer (e.g., PFA) end pieces (or “pucks”), by thermally bonding one end piece to each of the two opposed pleated ends of the pleated cylinder. The thermoplastic fluoropolymer end pieces and the laminating film at the opposed pleated ends (edges) of the pleated cylinder are softened by exposure to a heating element for 5 minutes (e.g., from 3 to 7 minutes), after 5 minutes the cartridge is lowered into the softened end pieces and the potting step is complete.

A cartridge, as described, including a polyimide membrane, can be included in a filter housing to form a filter product. The filter housing can be of any useful and desired size, shape, and materials, and can preferably be a fluorinated polymer such as a poly (tetrafluoroethylene-co-perfluoro(alkyvinylether)), TEFLON®, perfluoroalkoxyalkane (PFA), perfluoromethylalkoxy (MFA), or another suitable fluoropolymer (e.g., perfluoropolymer).

The membrane can be contained within a larger filter structure such as a filter housing or a filter cartridge that is used in a filtering system. The filtering system will place the membrane, e.g., as part of a filter or filter cartridge, in a flow path of a liquid chemical to cause at least a portion of the flow of the liquid chemical to pass through the membrane so that the membrane removes an amount of impurities or contaminants from the liquid chemical. The structure of a filter or filter cartridge may include one or more of various additional materials and structures (e.g., non-membrane filter structures) that support the membrane within the filter to cause fluid to flow from a filter inlet, through the membrane, and thorough a filter outlet, thereby passing through the membrane when passing through the filter.

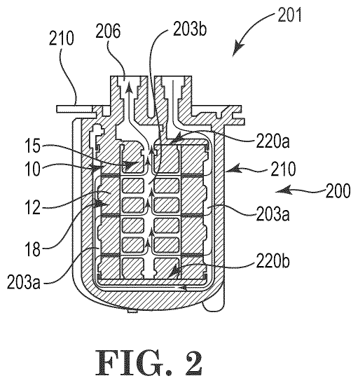

FIG. 2 illustrates, in cross section, an example of a fluid separation device or “filter” that includes a polyimide membrane of the present description in a filter assembly that includes non-membrane filter structures that are made entirely of fluoropolymer. The fluid separation device (filter) 200 includes housing 210, which contains polyimide membrane 12 at the interior. Membrane 12 includes two opposed edges located at each of two opposed pleated ends. Each pleated edge is thermally bonded to a thermoplastic fluoropolymer end piece 220a (top end piece) and 220b (bottom end piece) to form a fluid-tight seal between the edges of the pleated end and a surface of each flat end piece 220a, 220b. The thermally bonded edges at the pleated ends of membrane 12, i.e., the thermally bonded connection of the edge of the end pleated end to the flat end piece 220a, 220b, does not allow liquid to pass (leak) between the pleated end of membrane 12 and end piece 220a or 220b. Each connection between a thermally bonded end of a membrane 12 and the flat end piece 220a, 220b is therefore “fluid-tight.”

Preferred construction materials for the non-membrane filter structures of filter 200 of figure a filter include: PFA (perfluoroalkoxypolymer) as net supports (14 at FIG. 1A, not illustrated at FIG. 2); PFA for the housing 210, core 15, cage 18, top cap (top end piece) 220a, and bottom cap (bottom end piece) 220b; and FEP (perfluoro-ethylene-propylene polymer) as an edge lamination film connecting the membrane 12 to net supports 14 (not shown). No adhesive, e.g., potting compound, is required for the potting step, and a potting compound may be preferably excluded from the structures. The non-membrane filter structures can be constructed entirely of perfluorinated polymer, and all surfaces of the flow path between inlet 201 and outlet 206 that contact fluid (other than the polyimide membrane) are perfluorinated materials.

In use, a liquid feed enters the housing at opening 201 and is introduced to a first side of membrane 12 inside the housing. Membrane 12 separates the space within the housing into a first volume 203a and second volume 203b. The liquid “feed” introduced through inlet 201 to volume 203a contacts and passes through membrane 12 and enters volume 203b as a “permeate” that is the original feed after contaminants or impurities have been removed by membrane 12. The permeate exits volume 203b through outlet 206.

A filtration membrane as described herein, or a filter or filter component that contains the filtration membrane, can be useful in a method of filtering to purify or remove unwanted materials from a liquid chemical. The liquid chemical may be of any of various compositions, and may be a liquid chemical that is useful or used in any application, for any industrial or commercial use. Particular example of filters as described can be used to purifying a liquid chemical that is used or useful in a semiconductor or microelectronic fabrication application, e.g., for filtering a liquid solvent or other process solution used in a method of photolithography for semiconductor fabrication or processing (e.g., a liquid photoresist solution), a wet etching or cleaning step, a method of forming spin-on-glass (SOG), for a backside anti-reflective coating (BARC) method, etc.

The fluid can be any fluid, e.g., a solvent, that is required to exhibit a very high level of purity when used in a method of semiconductor photolithography, including a very low level of dissolved metals, and very low level of suspended particles or other impurities or contaminants. Some specific, non-limiting, examples of solvents that can be filtered using a filtration membrane as described include: n-butyl acetate (nBA), isopropyl alcohol (IPA), 2-ethoxyethyl acetate (2EEA), a xylene, cyclohexanone, ethyl lactate, gamma-butyrolactone, hexamethyldisilazane, methyl-2-hydroxyisobutyrate, methyl isobutyl carbinol (MIBC), n-butyl acetate, methyl isobutyl ketone (MIBK), isoamyl acetate, tetraethyl ammonium hydroxide (TMAH), propylene glycol monoethyl ether, propylene glycol methyl ether (PGME), 2-heptanone, and propylene glycol monomethyl ether acetate (PGMEA).

### EXAMPLES

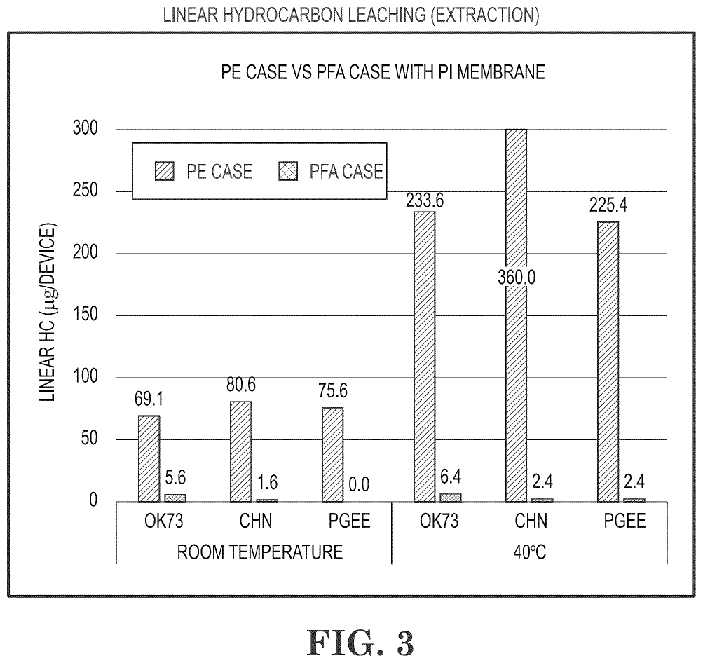

FIG. 3 shows performance data relating to liner hydrocarbon extraction from two filter products made using two different types of filter housings: PE housing (comparative) and PFA housing (inventive). Example filter PFA is made using a polyimide membrane as described and non-membrane filter structures made entirely of PFA. Comparative example filter PE is made using the same polyimide membrane and a non-membrane filter structures made of polyethylene. The data of the table at FIG. 3 shows that the PFA filter, having PFA non-membrane filter structures, exhibits substantially lower levels of hydrocarbon leaching using OK73 Thinner available from TOK America, cyclohexanone (CHN) and propylene glycol monoethyl ether (PGEE).

The test for linear hydrocarbon extraction was performed by filling each solvent in each filter device, leaving to stand at room temperature, collecting the solvent from the filter device after 24 hours, and then measuring extracted linear hydrocarbons with GC (Gas Chromatography). The filter devices were filled with each solvent again and left to stand at 40 degrees Celsius next for 24 hours. Linear hydrocarbons in the solvent were measured with GC.

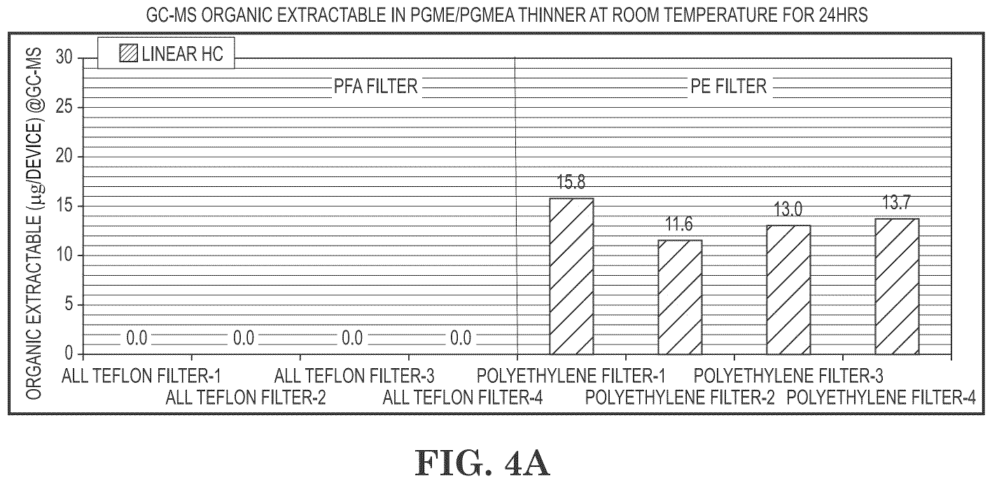

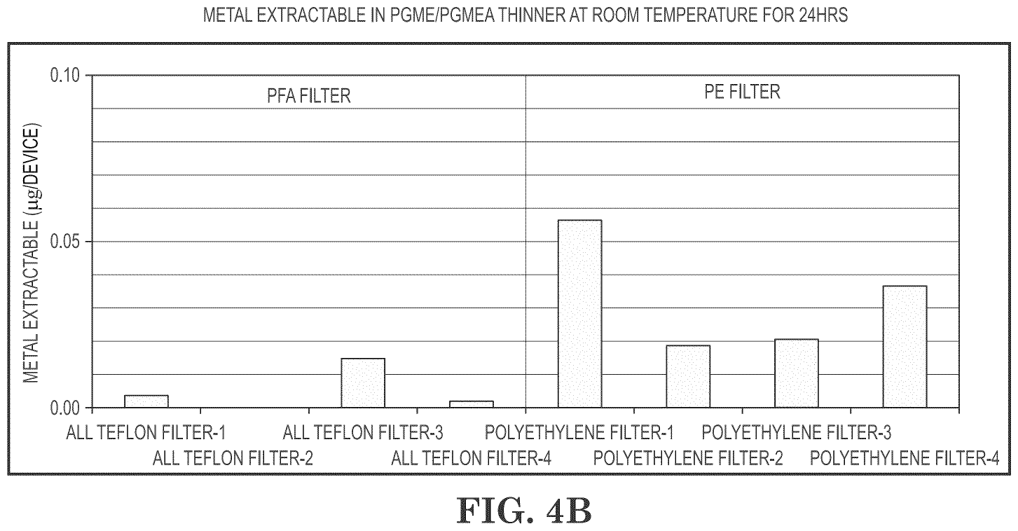

FIG. 4A shows performance data relating to organic extractables from two filter products made using two different types of filter housings: PE housing (comparative) and PFA housing (inventive). Example filter PFA is made using a polyimide membrane as described and non-membrane filter structures made entirely of PFA. Comparative example filter PE is made using the same polyimide membrane and a non-membrane filter structures made of polyethylene. The data of the table at FIG. 4A shows that the PFA filter, having PFA non-membrane filter structures, exhibits substantially lower levels of hydrocarbon leaching.

The test for linear hydrocarbon extraction was performed by filling each filter device with a combination of PGME and PGMEA, leaving to stand at room temperature, collecting the solvent from the filter device after 24 hours, and then measuring extracted linear hydrocarbons with GC (Gas Chromatography).

FIG. 4B shows performance data relating to metal extractables by testing two filter products made using two different types of filter housings: PE housing (comparative) and PFA housing (inventive). Example filter PFA is made using a polyimide membrane as described and non-membrane filter structures made entirely of PFA. Comparative example filter PE is made using the same polyimide membrane and a non-membrane filter structures made of polyethylene. The data of the table at FIG. 3 shows that the PFA filter, having PFA non-membrane filter structures, exhibits substantially lower levels of hydrocarbon leaching.

The test for metal extractables extraction was performed by each filter device with a combination of PGME and PGMEA, leaving to stand at room temperature, collecting the solvent from the filter device after 24 hours, and then measuring extracted metals using ICP-MS (Inductively Coupled Plasma-Mass Spectrometry). Table 1 below lists the results.

TABLE 1
Amount of Metal Extracted in μg/device)

| | PFA | PFA | PFA | PFA | PE | PE | PE | PE |
|---|---|---|---|---|---|---|---|---|
| Device | Filter 1 | Filter 2 | Filter 3 | Filter 4 | Filter 1 | Filter 2 | Filter 3 | Filter 4 |
| Li | 0 | 0 | 0 | 0 | 0 | 0 | 0 | 0 |
| Na | 0 | 0 | 0.0092 | 0 | 0.0247 | 0.0125 | 0.0132 | 0.0211 |
| Mg | 0 | 0 | 0 | 0 | 0.0039 | 0.0008 | 0.0006 | 0.0028 |
| Al | 0 | 0 | 0 | 0 | 0.0078 | 0 | 0 | 0.0010 |
| K | 0 | 0 | 0.0040 | 0.0011 | 0.0075 | 0.0013 | 0.0026 | 0.0045 |
| Ca | 0 | 0 | 0 | 0 | 0.0035 | 0.0038 | 0.0040 | 0.0061 |
| Ti | 0 | 0 | 0 | 0.0007 | 0.0037 | 0 | 0 | 0 |
| V | 0 | 0 | 0 | 0 | 0.0004 | 0 | 0 | 0 |
| Cr | 0 | 0 | 0 | 0 | 0 | 0 | 0 | 0 |
| Mn | 0 | 0 | 0 | 0 | 0 | 0 | 0 | 0 |
| Fe | 0 | 0 | 0.0012 | 0 | 0 | 0 | 0 | 0 |
| Co | 0 | 0 | 0 | 0 | 0.0001 | 0 | 0 | 0 |
| Ni | 0.0005 | 0 | 0 | 0 | 0.0005 | 0 | 0 | 0 |
| Cu | 0.0014 | 0 | 0 | 0 | 0.0003 | 0 | 0 | 0.0001 |
| Zn | 0 | 0 | 0 | 0.0002 | 0.0010 | 0 | 0 | 0 |
| Ge | 0.0005 | 0.0002 | 0.0005 | 0.0001 | 0 | 0 | 0 | 0 |
| As | 0.004 | 0 | 0 | 0 | 0 | 0 | 0 | 0.0002 |
| Sr | 0 | 0 | 0 | 0 | 0.0001 | 0 | 0 | 0.0001 |
| Mo | 0.0003 | 0 | 0 | 0 | 0 | 0 | 0 | 0 |
| Ag | 0 | 0 | 0 | 0 | 0 | 0 | 0 | 0 |
| Cd | 0 | 0 | 0 | 0.0003 | 0.0003 | 0 | 0.0002 | 0 |
| In | 0 | 0 | 0 | 0 | 0 | 0 | 0 | 0 |
| Sn | 0 | 0 | 0.0001 | 0 | 0 | 0 | 0 | 0 |
| Ba | 0 | 0 | 0 | 0 | 0.0010 | 0 | 0 | 0 |
| Ta | 0.0001 | 0 | 0 | 0 | 0.0007 | 0.0003 | 0 | 0.0006 |
| W | 0 | 0 | 0 | 0 | 0 | 0 | 0 | 0 |
| Au | 0.0004 | 0 | 0 | 0 | 0.0003 | 0 | 0 | 0 |
| Pb | 0 | 0 | 0 | 0 | 0.0003 | 0 | 0 | 0.0001 |

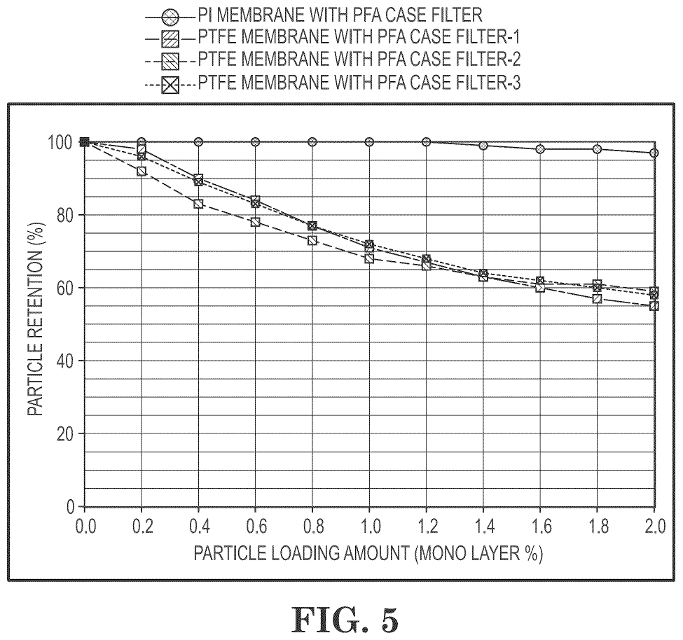

FIG. 5 shows performance data relating to particle removal efficiency (particle retention) by comparing four filter products: Polyimide Filter (inventive) and Filters 1, 2, and 3 (comparative). Example 1 (Polyimide Filter) is made using a polyimide membrane as described and non-membrane filter structures made entirely of PFA. Comparative example Filters 1, 2, and 3 are made using PTFE membranes and non-membrane filter structures made entirely of PFA.

“Particle retention” or “coverage” at FIG. 5 refers to the percentage of the number of particles that can be removed from a fluid stream by a membrane placed in the fluid pathway of the fluid stream. Particle retention of a sample filter membrane disc can be measured by passing a sufficient amount of an aqueous feed solution of 0.1% Triton X-100, containing 8 ppm polystyrene particles having a nominal diameter of 0.03 microns (available from Duke Scientific G25B), to achieve 1% monolayer coverage through a membrane at a constant flow of 7 mL/min, and collecting the permeate. The concentration of the polystyrene particles in the permeate can be calculated from the absorbance of the permeate. Particle retention is then calculated using the following equation:

$$particle retention = \frac{[ feed ] - [ filtrate ]}{[ feed ]} \times 100 \% .$$

The number (#) of particles necessary to achieve 1% monolayer coverage can be calculated from the following equation:

$$\# of particlesfor 1 \% monolayer = \frac{a}{\frac{\sqrt{3}}{2} {d}_{p}^{2}} \times \frac{1}{100}$$

where

$$a = effective membrane surface area$$

$${d}_{p} = diameter of the particle$$

“Nominal diameter,” as used herein, is the diameter of a particle as determined by photon correlation spectroscopy (PCS), laser diffraction or optical or SEM microscopy. Typically, the calculated diameter, or nominal diameter, is expressed as the diameter of a sphere that has the same projected area as the projected image of the particle. PCS, laser diffraction and optical microscopy techniques are well-known in the art.

In first aspect, a filter component comprises a porous filtration membrane comprising polyimide polymer and having an edge; and a support piece comprising thermoplastic fluoropolymer, wherein the edge is thermally bonded to the support piece to provide a fluid-tight seal between the edge and the support piece.

A second aspect according to the first aspect wherein, the edge is thermally bonded to the support piece by exposing the filtration membrane and the support piece to a temperature of at least 300 degrees Celsius for a time sufficient to soften the thermoplastic fluoropolymer.

A third aspect according to the first or second aspect, wherein the polyimide polymer has a tensile strength (machine direction) of at least 1000 mN per 5 mm and a tensile strength (cross direction) of at least 1000 mN per 5 mm.

A fourth aspect according to any preceding aspect, wherein the filtration membrane has a thickness in a range from 10 to 200 microns.

A fifth aspect according to any preceding aspect, wherein the filtration membrane exhibits: a bubble point in a range from 10 to 300 pounds per square inch measured using ethoxy-nonafluorobutane (HFE-7200) at a temperature of 25 degrees Celsius, an IPA flow time below 20,000 seconds per 500 milliliter measured at 21 degrees Celsius, or both.

A sixth aspect according to any preceding aspect, wherein the porous membrane contains at least 90 percent polyimide polymer.

A seventh aspect according to any preceding aspect, wherein the thermoplastic fluoropolymer is selected from the group consisting of poly (tetrafluoroethylene) (PTFE), poly (tetrafluoroethylene-co-hexafluoropropylene) (FEP), and poly (tetrafluoroethylene-co-perfluoro (alkylvinyl ether)) (FPA).

An eighth aspect according to any preceding aspect, wherein the filtration membrane is a sheet or a pleated sheet.

In a ninth aspect, a filter includes the filter component of any preceding aspect, the filter comprises: a fluoropolymer housing surrounding the filtration membrane, an inlet that allows fluid to flow into the housing, and an outlet that allows the fluid to flow out of the housing after the fluid passes through the membrane.

A tenth aspect according to the ninth aspect, comprising a flowpath defined by surfaces that are contacted by fluid flowing between the inlet and the outlet, wherein all surfaces of the flow path are made of fluoropolymer or the filtration membrane.

In an eleventh aspect, a method of using the filter of the ninth or tenth aspect, the method comprising passing fluid through the filtration membrane.

A twelfth aspect according to the eleventh aspect, wherein the fluid comprises solvent selected from the group consisting of: n-butyl acetate (nBA), isopropyl alcohol (IPA), 2-ethoxyethyl acetate (2EEA), a xylene, cyclohexanone, ethyl lactate, methyl isobutyl carbinol (MIBC), methyl isobutyl ketone (MIBK), isoamyl acetate, propylene glycol methyl ether (PGME, or (2-methoxy-1-methylethylacetate)), and propylene glycol monomethyl ether acetate (PGMEA), propylene glycol ethyl ether (PGEE), NMP (1-methyl-2-pyrrolidone), gamma-butyl lactone, dimethyl ether, dibutyl ether, and toluene.

A thirteenth aspect according to the eleventh aspect or the twelfth aspect, wherein the fluid comprises solvent selected from the group consisting of: propylene glycol methyl ether (PGME), propylene glycol monomethyl ether acetate (PGMEA), propylene glycol ethyl ether (PGEE), and cyclohexanone, and the filter exhibits a reduced amount of hydrocarbon leaching relative to a comparable filter containing the polyimide membrane and a polyethylene housing.

A fourteenth aspect according to the thirteenth aspect, wherein the filter exhibits an at least 50 percent reduction in hydrocarbon leaching relative to a comparable filter containing the polyimide membrane and a polyethylene housing.

In a fifteenth aspect, a method of preparing a filter component that includes a porous filtration membrane in contact with thermoplastic fluoropolymer, the porous filtration membrane comprising polyimide polymer and having an edge, the method comprises heating the thermoplastic fluoropolymer to soften the thermoplastic fluoropolymer.

A sixteenth aspect according to the fifteenth aspect, further comprising heating the thermoplastic fluoropolymer to a temperature of at least 400 degrees Celsius for a time sufficient to soften the thermoplastic fluoropolymer.

A seventeenth aspect according to the fifteenth or sixteenth aspect, wherein the thermoplastic fluoropolymer is an end piece, and the method comprises: exposing the filtration membrane and the thermoplastic fluoropolymer to a temperature of at least 400 degrees Celsius for a time sufficient to soften the thermoplastic fluoropolymer, and contacting the edge of the filtration membrane to the softened thermoplastic fluoropolymer, then reducing the temperature of the thermoplastic fluoropolymer to provide a fluid-tight seal between the edge and the end piece.

A nineteenth aspect according to any one of the fifteenth through seventeenth aspects, wherein the thermoplastic fluoropolymer is selected the group consisting of poly(tetrafluoroethylene) (PTFE), poly(tetrafluoroethylene-co-hexafluoropropylene) (FEP), and poly(tetrafluoroethylene-co-perfluoro(alkylvinyl ether)) (FPA).

1. A filter that includes a filter component which comprises:
a porous filtration membrane comprising polyimide polymer and having an edge, and
a support piece comprising thermoplastic fluoropolymer;
wherein the edge is bonded to the support piece to provide a fluid-tight seal between the edge and the support piece;
the filter comprising:
a fluoropolymer housing surrounding the porous filtration membrane,
an inlet that allows fluid to flow into the housing, and
an outlet that allows the fluid to flow out of the housing after the fluid passes through the porous filtration membrane.

2. The filter of claim 1, comprising a flowpath defined by surfaces that are contacted by fluid flowing between the inlet and the outlet, wherein all surfaces of the flow path are made of fluoropolymer or the filtration membrane.

3. The filter of claim 1, wherein the porous filtration membrane has a thickness in a range from 10 to 200 microns.

4. The filter of claim 1, wherein the filtration membrane exhibits:
a bubble point in a range from 10 to 300 pounds per square inch measured using ethoxy-nonafluorobutane (HFE-7200) at a temperature of 25 degrees Celsius,
an IPA flow time below 20,000 seconds per 500 milliliter measured at 21 degrees Celsius,
or both.

5. The filter component of claim 1, wherein the porous membrane contains at least 90 percent polyimide polymer.

6. The filter of claim 1, wherein the thermoplastic fluoropolymer of the filter component is selected from the group consisting of poly(tetrafluoroethylene) (PTFE), poly(tetrafluoroethylene-co-hexafluoropropylene) (FEP), and poly(tetrafluoroethylene-co-perfluoro(alkylvinyl ether)) (FPA).

7. A method of using the filter of claim 1, the method comprising passing fluid through the filtration membrane.

8. The method of claim 7, wherein the fluid comprises solvent selected from the group consisting of: n-butyl acetate (nBA), isopropyl alcohol (IPA), 2-ethoxyethyl acetate (2EEA), a xylene, cyclohexanone, ethyl lactate, methyl isobutyl carbinol (MIBC), methyl isobutyl ketone (MIBK), isoamyl acetate, propylene glycol methyl ether (PGME, or (2-methoxy-1-methylethylacetate)), and propylene glycol monomethyl ether acetate (PGMEA), propylene glycol ethyl ether (PGEE), NMP (1-methyl-2-pyrrolidone), gamma-butyl lactone, dimethyl ether, dibutyl ether, and toluene.

9. The method of claim 7, wherein:
the fluid comprises solvent selected from the group consisting of: propylene glycol methyl ether (PGME), propylene glycol monomethyl ether acetate (PGMEA), propylene glycol ethyl ether (PGEE), and cyclohexanone, and
the filter exhibits a reduced amount of hydrocarbon leaching relative to a comparable filter containing the polyimide membrane and a polyethylene housing.

10. The method of claim 9, wherein the filter exhibits an at least 50 percent reduction in hydrocarbon leaching relative to a comparable filter containing the polyimide membrane and a polyethylene housing.
